(19) United States
(12) Patent Application Publication
(10) Pub. No.: US 2023/0340618 A1
(43) Pub. Date: Oct. 26, 2023

(54) Method for Identifying Microorganisms and/or Their Resistance to Drugs, Cartridge and Related Cartridge Panel to Perform the Method

(71) Applicant: ALIFAX S.R.L., Polverara (IT)

(72) Inventor: Paolo Galiano, Padova (IT)

(73) Assignee: ALIFAX S.R.L., Polverara (IT)

(21) Appl. No.: 17/912,520

(22) PCT Filed: Mar. 18, 2021
(86) PCT No.: PCT/IT2021/050073

§ 371 Date: Sep. 18, 2022

(30) Foreign Application Priority Data
Mar. 19, 2020 (IT) 102020000005929

(51) Int. Cl. C12Q 1/689; C12Q 1/6895

(52) U.S. Cl. CPC C12Q 1/689; C12Q 1/6895; C12Q 2600/112; C12Q 2600/166; C12Q 2600/136

(57) ABSTRACT

A disposable cartridge suitable to carry out molecular biology tests starting from biological samples positive for microorganisms includes at least one specific reaction mixture for RT-PCR (Real Time Polymerase Chain Reaction) suitable for typing particular microorganisms and/or for providing information on relative resistance to antimicrobial and antifungal drugs, a panel that includes a plurality of cartridges and a method to identify at least one microorganism and/or its resistance to antimicrobial or antifungal drugs responsible for a systemic inflammatory state.

### FIELD OF THE INVENTION

Embodiments described here concern a method for the identification of microorganisms and/or their resistance to antimicrobial and antifungal drugs, a cartridge and a corresponding panel of cartridges to perform the method, wherein the microorganisms are present in a biological fluid, particularly in cases of generalized infection (sepsis).

### BACKGROUND OF THE INVENTION

It is known to treat subjects with infections from bacteria, filamentous and non-filamentous fungi, yeasts and molds, with suitable antimicrobial and/or antifungal therapy.

Antimicrobial and/or antifungal therapy, that is, the administration of one or more drugs able to counter the infection, is chosen by a healthcare professional, for example a doctor or clinician, generally on the basis of an identification of the type or types of microorganisms present in the infected subject, a test of their sensitivity to antibiotic or antifungal drugs and, optionally, any resistance of said microorganisms to antimicrobial or antifungal drugs.

Identification takes place on the basis of laboratory tests, or assays, that allow precise identification of the types of microorganisms, that is, they allow to type the group and/or species of microorganisms and to assess the in vitro sensitivity of these microorganisms to drugs. The effectiveness and promptness of administering the treatment to the patient depends on the speed and ease of performing said tests.

In some cases, the infection can be a risk to the patient's life, or cause serious health consequences even once it has been resolved, as can happen in cases where the infection is generalized and develops in the bloodstream.

In these cases, it is necessary to identify in the shortest possible time the type or types of microorganisms responsible and their possible in vitro sensitivity to antibiotic or antifungal drugs in order to promptly implement the most effective targeted therapy instead of the empirical and broad-spectrum therapy that is initially administered in order to stem the infection.

In fact, the more the administration of targeted and specific therapy is delayed, the greater the risk of serious adverse effects to the patient, such as the induction of drug resistance and the onset of severe septicemia, which may even cause the death of the subject.

The identification of the type of microorganisms responsible for the infection and their sensitivity to drugs is a phased process that can take up to 4 days to provide a reliable result, and requires highly specialized personnel.

The usual procedure provides that a test, for example a blood culture, is carried out as a first analysis on the blood sample of a patient suspected of systemic infection, which test defines the presence of potentially infectious microorganisms.

In the event of a positive result, a Gram stain test can be carried out to identify if the microorganisms responsible are a Gram-positive bacterium or a Gram-negative bacterium or, possibly, a fungus, a yeast, a mold or other.

The precise identification of the microorganisms, that is, the typing of family, genus and/or species and the detection of any resistance, takes place by means of a phenotypic examination in culture and, generally, after isolation in suitable growth media and/or with a genotypic examination using molecular biology techniques, such as for example Polymerase Chain Reaction (PCR) or Real Time PCR (RT-PCR or qPCR) which provide to amplify nucleic acids.

One disadvantage of this procedure is that the need to accurately identify the microorganisms from among dozens of types of microorganisms potentially responsible for the infection requires a very laborious preparation of the test, and therefore long times and specialized personnel.

The high manual component in the preparation of the test, or possibly tests, can cause errors that can lengthen the time required to obtain the result and/or alter the final results.

It is also known that microorganisms can be resistant to some antimicrobial or antifungal drugs, therefore, as well as identifying the microorganisms, it is also important to look for the presence of any possible resistance, in order to avoid administering an ineffective therapy. Such research can be carried out by genotypic analyses, such as RT-PCR to identify resistance to antibiotics or antifungals, and/or phenotypic analyses as a test of sensitivity to antibiotics or antifungals, but it is clear that this further analysis, which is necessary on most occasions, lengthens the time and complexity of the procedure.

The articles by Anne J. Blaschke et al, “Rapid Identification of pathogen . . . ” and by Mark Poritz et al. “Film-array, an automatic Multiplex PCR System . . . ” describe the use of disposable bags comprising frozen reagents to carry out a two-step real-time PCR analysis. PCR amplification is carried out in separate wells. Each well contains specific frozen primers deriving from a first PCR amplification step. These documents describe solutions using FilmArray® technology, with frozen reagents.

WO 2019/040769 and the article by Te-Din Huang et al. describe identification panels for cell cultures based on real-time PCR analysis for the identification of resistant bacteria, fungi and genes. The panel comprises disposable cartridges, which include oligonucleotides and reagents to carry out a nucleic acid amplification reaction. However, these systems are based on electrochemical detection, not fluorescence detection.

There is therefore a need to perfect a method and produce corresponding cartridges to identify microorganisms and/or their resistance to antimicrobial or antifungal drugs that can overcome at least one of the disadvantages of the state of the art.

In particular, one purpose of the present invention is to provide a method, a panel of cartridges and the corresponding cartridges, to carry out the rapid identification of microorganisms and/or corresponding resistance to antimicrobial or antifungal drugs that reduce the times needed to obtain a result.

Another purpose is to provide a method, a panel of cartridges and the corresponding ready-to-use cartridges that reduce the need for specialized personnel, that can be easily used by departments that are open 24 hours a day and in peripheral laboratories.

Another purpose of the present invention is to provide a method, a panel of cartridges and the corresponding cartridges usable in known PCR or RT-PCR instruments.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes, the invention provides a method, a cartridge for carrying out the method, and a corresponding panel of cartridges, for the identification of microorganisms and/or their resistance to drugs, wherein the microorganisms are present in a biological fluid, in particular in the case of a generalized infection (sepsis).

According to the invention, it is provided to supply a disposable cartridge suitable to carry out molecular biology tests starting from biological samples positive for microorganisms, the cartridge comprising at least one specific reaction mixture for Real Time PCR (RT-PCR or qPCR) suitable for typing particular microorganisms and/or providing information on relative resistance to antimicrobial and/or antifungal drugs, wherein such tests are carried out by means of thermal cycles combined with the acquisition of fluorescent signals of specific reagents.

According to the invention, the method comprises the following steps:
preparing a plurality of cartridges each provided with at least one specific reaction mixture for RT-PCR, advantageously a plurality of reaction mixtures for RT-PCR suitable for typing particular microorganisms and/or providing information on relative resistance to antimicrobial or antifungal drugs, wherein each of the cartridges allows to carry out molecular biology tests starting from biological samples positive for microorganisms;
supplying a biological sample positive for the presence of at least one microorganism responsible for the generalized infection, the classification of which is preferably known, derived from a microscopic picture that comprises Gram positive bacteria, Gram negative bacteria, yeast or filamentous and non-filamentous fungi, molds;
selecting at least one specific cartridge from the plurality of cartridges on the basis of the classification deriving from the microscopic picture of the microorganism;
carrying out at least one molecular biology analysis using RT-PCR with at least one selected cartridge, wherein the positive biological sample preferably consists of samples deriving from blood culture positive for the presence of at least one microorganism.

The positive biological sample can also include:
purified DNA/RNA from biological sample with a presumed positivity for at least one microorganism; or
single colony of a microorganism, intended as bacterial growth, originating from a biological sample.

The disposable cartridge is of the type developed with MEMS (Micro Electro-Mechanical Systems) technology which, thanks to accurate thermal cycles combined with the acquisition of fluorescent signals of specific reagents, allows to carry out the molecular biology tests as above.

A plurality of cartridges as identified above form a panel from which the operator selects, on each occasion, the suitable cartridge based on the results of the previous operations, such as Gram staining or similar techniques.

The peculiarity of the present invention is that it allows the correct and rapid identification of pathogenic microorganisms and their susceptibility to pharmacological treatments, such as antibiotics or antifungals, in very short analysis times, even shorter than an hour. This characteristic is of extreme importance, especially in dealing with an extremely serious clinical picture such as sepsis, in which reporting times can significantly affect the reduction in mortality rate.

In fact, once the classification deriving from the microscopic picture of the microorganism responsible for a generalized infection, in particular of the blood, is known, the invention allows to carry out more targeted analyzes, based only on the classification deriving from the microscopic picture detected.

In particular, with the present invention, for the state of sepsis, Gram staining or similar techniques are used as an indication for the choice of the cartridge(s).

A technique similar to Gram staining can be, for example, the L-Alanine Aminopeptidase test.

According to the invention, a panel is also provided for the identification of microorganisms and/or their resistance to antimicrobial drugs. The panel comprises a plurality of cartridges, each comprising at least one well inside which there is a reaction mixture for RT-PCR, wherein at least one mixture contained in each cartridge is specific for a classification deriving from the microscopic picture of a microorganism.

The present invention falls into the category of clinical emergency tests and tests on specific cases and Point of Care.

With the present invention it is possible to detect up to 36 different nucleotide sequences, or targets, in a single cartridge. Each of the cartridges contains at least one positive control and one negative control for each sample being analyzed, in order to verify the functioning of the cartridge itself and to evaluate the presence of inhibitors in the single biological sample.

### BRIEF DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a schematic representation from above of a cartridge for use in an RT-PCR tool for carrying out a method in accordance with some embodiments described here;

FIG. 2 is a schematic representation from above of a panel of cartridges for carrying out a method in accordance with some embodiments described here.

To facilitate comprehension, the same reference numbers have been used, where possible, to identify identical common elements in the drawings. It is understood that elements and characteristics of one embodiment can conveniently be incorporated into other embodiments without further clarifications.

### DETAILED DESCRIPTION OF SOME EMBODIMENTS

We will now refer in detail to the possible embodiments of the invention and to a possible application thereof described by the examples provided. Each example is supplied by way of illustration of the invention and shall not be understood as a limitation thereof. For example, one or more characteristics shown or described insomuch as they are part of one embodiment can be varied or adopted on, or in association with, other embodiments to produce another embodiment. It is understood that the present invention shall include all such modifications and variants.

Before describing these embodiments, we must also clarify that the present description is not limited in its application to details of the construction as described in the following description using the attached examples. The present description can provide other embodiments and can be obtained or executed in various other ways. We must also clarify that the phraseology and terminology used here is for the purposes of description only, and cannot be considered as limitative.

The following invention concerns a method, a cartridge and a corresponding panel of cartridges for carrying out the method, for the identification of microorganisms and/or their resistance to drugs, wherein the microorganisms are present in a biological fluid, in particular in case of a generalized infection (sepsis).

With the term “microorganism(s)” we mean bacteria, filamentous and non-filamentous fungi, yeasts or molds.

With the term “typing” or “typed” we mean the classification of a microorganism by family, genus and/or species.

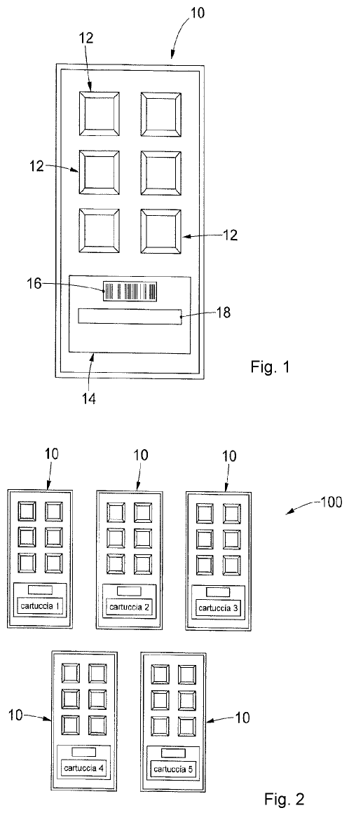

With reference to FIG. 1, a disposable cartridge is shown suitable to carry out molecular biology tests starting from biological samples positive for at least one microorganism, comprising at least one specific reaction mixture for RT-PCR (Real Time Polymerase Chain Reaction) suitable for typing particular microorganisms and/or providing information on relative resistance to antimicrobial and/or antifungal drugs, wherein such tests are carried out by means of thermal cycles combined with the acquisition of fluorescent signals of specific reagents.

According to the invention, the method comprises:
preparing a plurality of cartridges 10, each provided with at least one reaction mixture for RT-PCR, advantageously a plurality of reaction mixtures for RT-PCR suitable for typing microorganisms and/or providing information on relative resistance to antimicrobial or antifungal drugs, wherein each of the cartridges 10 allows to carry out molecular biology tests starting from biological samples positive for microorganisms;
supplying a biological sample positive for the presence of microorganisms responsible for the generalized infection, the classification of which is known, derived from the microscopic picture that comprises: Gram positive bacteria, Gram negative bacteria, yeast or filamentous and non-filamentous fungi, molds;
selecting at least one specific cartridge 10 from the plurality of cartridges 10 on the basis of the classification deriving from the microscopic picture of the microorganism;
carrying out at least one molecular biology analysis using RT-PCR with the at least one cartridge 10 selected, wherein the positive biological sample preferably consists of samples deriving from blood culture positive for the presence of at least one microorganism.

The biological sample can also include:
purified DNA/RNA from a biological sample with a presumed positivity for at least one microorganism; or
single colony of a microorganism, intended as bacterial growth, originating from a biological sample.

The preparation of the biological sample is specific to the starting sample and is as follows.

In the case of a positive blood culture, the steps are the following:
taking a volume (by way of example from 50 to 500 μl) of the positive blood culture, centrifugation at a speed comprised between 200 g and 700 g from 20 seconds to 5 minutes, removal of the supernatant and centrifugation thereof at a speed comprised between 2000 g and 7000 g from 20 seconds to 5 minutes;
elimination of the supernatant and resuspension of the pellet in a volume of ultra-pure water (by way of example, between 500 and 1500 μL) free of DNase and RNase. The resuspension thus obtained is used for PCR or RT-PCR.

An alternative method to recover microorganisms starting from positive blood culture can provide to transfer the biological sample into serum separator tubes, with or without additives (including gelatinous, accelerator, activator materials and/or granules) which, following centrifugation, allow to separate and then collect microorganisms.

In the case of a colony of microorganisms, the procedure provides to remove one colony and dilute it in a volume of ultra-pure water (by way of example, between 100 and 2000 μL) free of DNase and RNase.

In the case of suspension of microorganisms, the procedure can provide to use the suspension itself or, possibly, after adjusting the concentration of microorganisms contained therein.

In the case of nucleic acids, the DNA/RNA can be extracted from a biological sample with a commercially available kit dedicated to the type of starting biological sample, or from an automated system for the extraction.

In clinical practice, a classification deriving from the microscopic picture is routinely carried out on positive blood culture samples, by means of Gram staining or similar technique.

The classification deriving from the microscopic picture of the microorganism can be Gram positive bacterium, Gram negative bacterium, or yeast or fungus, or mold.

Possibly, the classification can be carried out with the L-Alanine Aminopeptidase test or similar tests.

According to the invention, once the classification deriving from the microscopic picture of the microorganisms has been identified, the healthcare worker can, on the basis of this identification, conduct a targeted investigation by putting the positive biological sample in contact with the predefined cartridge for that specific classification deriving from the microscopic picture.

In the same way, it is possible to put the positive biological sample in contact with a suitable cartridge in order to obtain information on the possible resistances of the microorganisms.

According to some embodiments, the method provides to put the biological sample in contact, without the step of extracting nucleic acids.

Advantageously, this facilitates the analysis, reducing time and personnel with specific skills.

According to some embodiments, the reaction mixture for RT-PCR comprises, for example, a buffer, polymerase enzymes, sets of amplification primer pairs and probes.

In accordance with some embodiments, a reaction mixture comprises up to six sets of different primers and probes, each suitable to allow the amplification of respective different gene sequences.

According to some embodiments, the amplification primers allow to amplify nucleotide sequences which can be gene sequences suitable for typing microorganisms and/or detecting resistance to one or more antimicrobial or antifungal drugs.

The nucleotide sequences can be genomic or plasmid DNA or cDNA or RNA.

The genes that confer the resistances can be, for example, Van A, Van B, Van C1, Van C 2/3, Mec A, Mec B, Mec C, SCCmec orf X for Gram positive bacteria, CTX-M, SHV, TEM, Colistin mcr 1, Colistin mcr 2, Colistin mcr-3, Colistin mcr-4, Colistin mcr-5, GES, IMP, NDM, OXA 23, CMY-1/MOX, KPC, OXA 48, VIM, CMY-II, PER, FIM, for Gram negative bacteria, resistance to antifungals (such as Cyp51, ERG11) for yeasts and fungi.

In accordance with some embodiments, the detection of microorganisms and resistances can be achieved in separate reaction mixtures or in the same mixture.

Some embodiments can provide to put the biological fluid in contact with a series of mixtures of equal composition in order to provide replicates.

The method provides to use probes complementary to the amplified products associated with a fluorophore able to emit a detectable signal.

In order to recognize different amplified products in the same mixture, it is provided to use probes complementary to one or the other product, which are associated with different fluorophores.

The fluorophore can be selected from a group comprising Rhodamine-X (ROXTM), Hexachloro-fluorescein (HEXTM), NEDTM, Fluorescein (FAMTM), SYBRTM Green, VICTM, TAMRATM, ATTOTM, TETTM, JOE, Texas Red®, Cy3TM, Cy5TM, Cy5.5 TM or analogues thereof.

According to some embodiments, multiple tools can be used simultaneously to carry out RT-PCR analyzes in order to perform multiple analyzes independently, and allowing to analyze multiple cartridges in less than an hour starting from the loading of the sample on the cartridge.

In some embodiments, the method can provide to personalize the cartridge with information specific to it, such as, for example, an identifier of the reagents, and of the test to be performed, compatibility with the instrument and the software, batch, expiration date by means of technologies such as RFID (Radio Frequency Identification).

In some embodiments, the method can also provide a report identifying the microorganisms detected and any resistances based on the amplified sequences in the biological fluid.

According to some embodiments, the report can be viewed on an interface of a computer or a smart mobile device.

In some embodiments, the results can possibly be used by the computer to define the correct and effective antibiotic or antifungal therapy.

For example, the results of the report can be compared with a database that associates the microorganisms identified and/or any resistance with the most effective drug(s).

With reference to FIG. 2, each cartridge 10 comprises a plurality of wells 12, each containing a reaction mixture for RT-PCR.

In preferred embodiments, the reaction mixtures are lyophilized or gelled or dried or treated with similar methods.

A reaction mixture can allow to identify one or more target nucleotide sequences, for example two or three or four, up to six.

According to some embodiments, the wells 12 of the cartridge 10 can be a number comprised between 1 and 24, preferably between 2 and 12, even more preferably between 4 and 8, even more preferably 6.

According to some embodiments, a cartridge with six wells, each containing a reaction mixture for RT-PCR that allows to amplify up to six different nucleotide sequences, can allow to amplify up to thirty-six different nucleotide sequences.

In accordance with some embodiments, the cartridge 10 allows to obtain information on the typing and/or information on resistance to antimicrobial or antifungal drugs of a specific classification deriving from the microscopic picture.

According to some embodiments, the classification resulting from the microscopic picture can be selected from a group comprising: Gram positive bacteria, Gram negative bacteria, fungi, yeasts or molds.

According to some embodiments, the cartridge 10 is of the type developed with MEMS (Micro Electro-Mechanical Systems) technology.

Advantageously, these cartridges allow, if used in suitable instruments, to obtain results in less than an hour and with a minimum reaction volume from 5 to 30 μL.

In some embodiments, the cartridge 10 can comprise at least one recognition mean 14.

The recognition mean 14 can provide information specific to the cartridge 10, such as, for example, an identifier of the reagents, compatibility with the instrument and the software, batch, expiry date, etc.

The recognition mean 14 can comprise, for example, a barcode 16 or an RFID (Radio Frequency Identification).

To facilitate the recognition of the cartridge 10 for a user, an identifier 18 can be present, which can show the name of the type of cartridge.

Optionally, a color code, or a code that makes it even easier to recognize the cartridge 10, can be used.

An advantageous application of the invention is in identifying microorganisms causative of generalized or systemic infection (sepsis), and their possible resistance to drugs.

The invention provides, by way of example, a panel comprising at least three, advantageously five, cartridges for the predefined typing of microorganisms known to cause sepsis and/or their resistances.

An expert in the field will note how each predefined typing set is carefully chosen to provide an exhaustive detection of microorganisms and/or resistances for the cases proposed and, intuitively, with the execution of a single RT-PCR operation.

For example, a set of primers “A” recognizes the group of bacteria “a” which comprises the species “x” of which there is, inside the cartridge, a specific set “X” suitable for its detection, however, the set “A” also recognizes the species “y”, which is not recognized by the specific set of primers “X” inside the cartridge.

Therefore, the invention allows to significantly expand the number of species of microorganisms that can be diagnosed with a single cartridge.

It is therefore clear that the invention allows, even in an emergency, to obtain complete information quickly.

The invention also provides a panel 100 comprising a plurality of cartridges 10 including at least, as a definition of minimum panel, the following:
a) A cartridge for the identification of Gram negative and/or their resistance to antibiotics,
b) A cartridge for the identification of Gram positive and/or their resistance to antibiotics,
c) A cartridge for the identification of yeasts, filamentous and non-filamentous fungi, molds and/or their resistance to antifungals.

With reference to FIG. 2, an example panel 100 is depicted comprising five cartridges for the identification of microorganisms and/or their resistance to antimicrobial or antifungal drugs responsible for a generalized infection. The panel 100 in question comprises: a cartridge for the typing of Gram positive microorganisms, in particular staphylococci and their resistances, a cartridge for the typing of non-staphylococci Gram positive microorganisms and their resistances, a cartridge for the typing of Gram negative microorganisms, a cartridge for the identification of resistances of Gram negative microorganisms, a cartridge for the typing of yeasts, filamentous and non-filamentous fungi, molds and/or their resistance to antifungals.

In one variant, the panel 100 can comprise a sixth cartridge for genes that confer resistance to colistin.

In another variant, the cartridge that can be used for typing and searching for resistances of yeasts, fungi and molds can be divided into additional cartridges, identified below as 5a-5d.

The cartridges are detailed in the panel example described below.

If a systemic infection caused by Gram positive bacteria is detected, it is advantageous to put the biological fluid in contact with the set of the cartridge 1 or 2, as appropriate.

Conversely, in case of systemic infection caused by Gram negative bacteria, it is advantageous to put the biological fluid in contact with the set of the cartridge 3 and/or cartridge 4, as appropriate.

Possibly, in the event that the systemic infection can be traced back to an infection from unknown yeasts or fungi or molds, it is advantageous to put the biological fluid in contact with one or more of the sets provided in the cartridges 5a-5d.

The panel 100 can in any case comprise any combination whatsoever of the examples described or other specific cartridges for other microorganisms or their resistances.

Each cartridge also provides the amplification of at least one gene sequence used as a positive control and the absence of amplification of at least one gene sequence as a negative control of the RT-PCR reaction.

The invention therefore provides a method, a cartridge to carry out the method and a corresponding panel of cartridges which advantageously offers a user the possibility, once the result of the Gram staining and therefore the classification deriving from the microscopic picture, or the result of a similar technique, such as for example the L-Alanine Aminopeptidase test, has been obtained, of selecting the cartridge(s) suitable to perform, on one or more RT-PCR instruments compatible with the panel of cartridges, a targeted analysis to identify the family and/or the group and/or the species of microorganisms and any relative resistance to drugs thereof, in an intuitive, practical and fast manner.

The following are specifications relating to the nine (total) example cartridges identified above, which can form a panel of cartridges. For each of them, the list of corresponding identifiable microorganisms and/or resistances is provided.

### Panel Example

1. A cartridge (cartridge 1) allows the identification, by way of example, of Staphylococcus Gram positive bacteria and the relative resistances, differentiating 9 Staphylococcus Gram positive bacterial species.

“Staphylococcus Typing and Resistances” Set

Cartridge 1 Example
Staphylococcus spp
Staphylococcus aureus
Staphylococcus epidermidis
Staphylococcus haemolyticus
Staphylococcus hominis
Staphylococcus lugdunensis
Staphylococcus saprophyticus
Staphylococcus sciuri
Staphylococcus simulans
Staphylococcus xylosus
Mec A
Mec C
SCC mec-orfX
Van A
Van B
Positive control
Negative control

2. A cartridge (cartridge 2) allows the identification, by way of example, of 10 different Gram positive bacterial species belonging to the Streptococcaceae, Enterococcaceae, Bacillaceae, Listeriaceae family and relative resistance to antibiotics.

“Non-staphylococcus Gram Positive Typing and Resistances” Set

Cartridge 2 Example
Bacillus subtilis
Enterococcus spp
Enterococcus faecalis
Enterococcus faecium
Listeria monocytogenes
Streptococcus spp
Streptococcus agalactiae
Streptococcus anginosus
Streptococcus pneumoniae
Streptococcus pyogenes
VanA
Van B
VanC1
VanC 2/3
Positive control
Negative control

3. A cartridge (cartridge 3) allows to discriminate up to, by way of example, 16 different Gram negative bacterial species.

“Gram Negative Typing” Set

Cartridge 3 Example
Acinetobacter baumannii
Enterobacteriaceae
Enterobacter cloacae complex
Escherichia coli
Haemophilus influenzae
Klebsiella aerogenes
Klebsiella oxytoca
Klebsiella pneumoniae
Neisseria meningitidis
Proteus spp
Proteus mirabilis
Pseudomonas aeruginosa
Salmonella typhi
Serratia marcescens
Shigella spp
Stenotrophomonas maltophilia
Positive control
Negative control

4. A cartridge (cartridge 4) that allows the identification of, by way of example, 15 gene sequences correlated with a drug resistance phenotype.

Resistances: Carbapemenase, ESBL, AmpC, Colistin

Gene sequences: KPC, NDM, VIM, OXA-48, OXA-23, IMP, GES, TEM, CTX-M, SHV, CMY-I/MOX, CMY2, mcr-1, mcr-2, mcr-4.

“Gram Negative Resistances” Set

Cartridge 4 Example
CTX-M
SHV
TEM
Colistin mcr-1
Colistin mcr-2
Colistin mcr-4
GES
IMP
NDM
OXA-23
CMY-I/MOX
KPC
OXA-48
VIM
CMY-II
Positive control
Negative control

5. A cartridge (cartridge 5) allows the identification of yeasts, filamentous and non-filamentous fungi, and molds and/or relative resistances; this cartridge can comprise one or more of the four different configurations (from 5a to 5d) of detectable targets, by way of example, as reported below.

“Yeasts, Filamentous and Non-Filamentous Fungi and Molds Typing and Resistances” Set

Cartridge 5a Example
Aspergillus spp
Aspergillus fumigatus
Aspergillus vescicular
Aspergillus nidulans
Aspergillus terreus
Aspergillus flavus
Aspergillus niger
Candida spp
Cyp51A (ERG11)
Positive control
Negative control

Cartridge 5b Example
Candida spp
Candida albicans
Candida auris
Candida glabrata
Candida krusei
Candida parapsilosis
Candida tropicalis
Candida lusitaniae
Candida dubliniensis
Candida guilliermondii
Positive control
Negative control

Cartridge 5c Example
Candida spp
Zygomycetes (Mucorales, entomophthorales)
Fusarium spp
Cryptococcus spp
Pneumocystis
Aspergillus spp
Positive control
Negative control

Cartridge 5d Example
Candida spp
Blastomyces
Coccidioides
Histoplasma
Paracoccidioides
Talaromyces (Penicillium)
Sporothrix
Emmonsia
Positive control
Negative control

Optionally, an additional cartridge (Cartridge 6) can be provided as follows, to allow the targeted identification of those targets that identify colistin resistance.

Cartridge 6 Example
Mcr-1
Mcr-2
Mcr-3
Mcr-4
Mcr-5
Positive control
Negative control.

It is clear that modifications and/or additions of steps may be made to the identification method, to the cartridge and to the corresponding panel of cartridges as described heretofore, without departing from the field and scope of the present invention as defined by the claims.

It is also clear that, although the present invention has been described with reference to some specific examples, a person of skill in the art shall certainly be able to achieve many other equivalent forms of identification method, cartridge and panel of cartridges, having the characteristics as set forth in the claims and hence all coming within the field of protection defined thereby.

1. A disposable cartridge suitable to carry out molecular biology tests starting from biological samples positive for microorganisms, comprising at least one specific reaction mixture for RT-PCR (Real Time Polymerase Chain Reaction) suitable for typing particular microorganisms and/or for providing information on relative resistance to antimicrobial and/or antifungal drugs, wherein said tests are carried out by means of thermal cycles combined with the acquisition of fluorescent signals of specific reagents, wherein said cartridge is made with MEMS (Micro Electro-Mechanical Systems) technology and is selectively suitable at least for:
a) identification of Gram-positive bacteria belonging to the Staphylococcaceae family and/or relative resistance to antibiotics, or
b) identification of Gram-positive bacteria not belonging to the Streptococcaceae family and/or relative resistance to antibiotics, or
c) identification of Gram-negative bacteria, or
d) identification of resistance to antibiotics in Gram-negative bacteria, or
e) identification of yeasts, filamentous and non-filamentous fungi, molds and/or their resistance to antifungals.

2. The disposable cartridge as in claim 1, containing at least one positive control and one negative control to verify the functioning of the cartridge itself and to evaluate the presence of inhibitors in the single biological sample.

3. The disposable cartridge as in claim 1, containing reaction mixtures for detecting up to 36 different nucleotide sequences.

4. The disposable cartridge as in claim 1, wherein one or more of the reaction mixtures for RT-PCR are lyophilized o subjected to gelling, drying or treated with similar preserving procedures.

5. The disposable cartridge as in claim 1, wherein the cartridge suitable for the identification of Gram-positive bacteria belonging to the Staphylococcaceae family and relative resistance to antibiotics comprises the following targets: Staphylococcus spp, Staphylococcus aureus, Staphylococcus epidermidis, Staphylococcus haemolyticus, Staphylococcus hominis, Staphylococcus lugdunensis, Staphylococcus saprophyticus, Staphylococcus sciuri, Staphylococcus simulans, Staphylococcus xylosus, Mec A, Mec C, SCC mec-orfX, Van A, Van B, Positive control, Negative control.

6. The disposable cartridge as in claim 1, wherein the cartridge suitable for the identification of Gram-positive bacteria belonging to the Streptococcaceae, Enterococcaceae, Bacillaceae, Listeriaceae family and/or relative resistance to antibiotics comprises the following targets: Bacillus subtilis, Enterococcus spp, Enterococcus faecalis, Enterococcus faecium, Listeria monocytogenes, Streptococcus spp, Streptococcus agalactiae, Streptococcus anginosus, Streptococcus pneumoniae, Streptococcus pyogenes, VanA, VanB, VanC1, VanC 2/3, Positive control, Negative control.

7. The disposable cartridge as in claim 1, wherein the cartridge suitable for the identification of Gram-negative bacteria comprises the following targets: Acinetobacter baumannii, Enterobacteriaceae, Enterobacter cloacae complex, Escherichia coli, Haemophilus influenzae, Klebsiella aerogenes, Klebsiella oxytoca, Klebsiella pneumoniae, Neisseria meningitidis, Proteus spp, Proteus mirabilis, Pseudomonas aeruginosa, Salmonella typhi, Serratia marcescens, Shigella spp, Stenotrophomonas maltophila, Positive control, Negative control.

8. The disposable cartridge as in claim 1, wherein the cartridge suitable for the identification of resistance to antibiotics in Gram-negative bacteria comprises the following targets: CTX-M, SHV, TEM, Colistin mcr-1, Colistin mcr-2, Colistin mcr-4, GES, IMP, NDM, OXA-23, CMY-I/MOX, KPC, OXA-48, VIM, CMY-II, PER, FIM, Positive control, Negative control.

9. The disposable cartridge as in claim 1, wherein the cartridge suitable for the identification of yeasts, filamentous and non-filamentous fungi, molds and/or their resistance to antifungals comprises one or more targets belonging to the following target groups:
a) Aspergillus spp, Aspergillus fumigatus, Aspergillus vescicular, Aspergillus nidulans, Aspergillus terreus, Aspergillus flavus, Aspergillus niger, Candida spp, Cyp51A (ERG11), Positive control, Negative control;
b) Candida spp, Candida albicans, Candida auris, Candida glabrata, Candida krusei, Candida parapsilosis, Candida tropicalis, Candida lusitaniae, Candida dubliniensis, Candida guilliermondii, Positive control, Negative control;
c) Candida spp, Zygomycetes (Mucorales, Entomophthorales), Fusarium spp, Cryptococcus spp, Pneumocystis, Aspergillus spp, Positive control, Negative control;
d) Candida spp, Blastomyces, Coccidioides, Histoplasma, Paracoccidioides, Talaromyces (Penicillium), Sporothrix, Emmonsia, Positive control, Negative control.

10. The disposable cartridge as in claim 1, wherein the cartridge suitable for the identification of genes that code for resistance to colistin comprises: Mcr-1, Mcr-2, Mcr-3, Mcr-4, Mcr-5, Positive control, Negative control.

11. A panel comprising a plurality of cartridges (10) as in claim 1, comprising at least:
a) a cartridge for the identification of Gram-negatives and/or their resistance to antibiotics,
b) a cartridge for the identification of Gram-positives and/or their resistance to antibiotics,
c) a cartridge for the identification of yeasts, filamentous and non-filamentous fungi, molds and/or their resistance to antifungals.

12. A method to identify at least one microorganism and/or its resistance to antimicrobial or antifungal drugs responsible for a systemic inflammatory state, in particular a generalized infection, said method comprising the following steps:
preparing a plurality of disposable cartridges as in claim 1, each provided with at least one specific reaction mixture for RT-PCR suitable for typing particular microorganisms and/or providing information on relative resistance to antimicrobial or antifungal drugs, wherein each of the cartridges allows to carry out molecular biology tests starting from biological samples that are positive for microorganisms;
supplying a biological sample positive for the presence of microorganisms responsible for generalized infection, the classification of which is preferably known, derived from the microscopic picture,
selecting at least one specific cartridge (10) from said plurality of cartridges (10) on the basis of the classification deriving from the microscopic picture of the microorganism,
carrying out at least one molecular biology analysis using RT-PCR with said at least one cartridge (10) selected, wherein the positive biological sample consists of samples deriving from a blood culture positive for the presence of at least one microorganism.

13. The method as in claim 12, wherein if the biological sample is a blood culture positive for the presence of a microorganism, preparing the sample comprises the following steps:
taking a volume, advantageously between 50 and 500 μl, of the positive blood culture; centrifugation at a speed comprised between 200 g and 700 g from 20 seconds to 5 minutes, removal of the supernatant, and centrifugation thereof at a speed comprised between 2000 g and 7000 g from 20 seconds to 5 minutes, elimination of the supernatant and resuspension of the pellet in a volume of ultra-pure water, advantageously between 500 and 1500 μL, free of DNase and RNase, and use of the suspension thus obtained for PCR or RT-PCR.

14. The method as in claim 12, wherein if the biological sample is a blood culture positive for the presence of a microorganism, the preparation of the sample occurs by means of treatment techniques comprising separation systems for obtaining microorganisms or nucleic acids or similar material suitable for molecular tests.

15. The method as in claim 12, where as biological sample the following are also included:
purified DNA/RNA from a biological sample with a presumed positivity for at least one microorganism; or
single colony of a microorganism, intended as bacterial growth, originating from a biological sample.

16. The method as in claim 15, wherein in the case of a colony of microorganisms it provides the removal of a colony and the dilution thereof in a volume of ultra-pure water free of DNase and RNase.

17. The method as in claim 15, wherein in the case of nucleic acids, the DNA/RNA can derive from extraction from a biological sample with a commercially available kit, or from automated systems dedicated to the type of the starting biological sample.

18. The method as in claim 12, wherein the analysis to identify the classification deriving from the microscopic picture of the microorganisms is carried out by means of Gram staining or L-Aminopeptidase or similar.

19. The method as in claim 12, wherein the typing provides information on the family and/or genus and/or species of the microorganisms and/or relative resistance.
